(19) United States
(12) Patent Application Publication
(10) Pub. No.: US 2010/0268235 A1
(43) Pub. Date: Oct. 21, 2010

(54) DEVICE FOR CREATING A SCREW-SHAPED, PARTICULARLY WORM-SHAPED RECESS IN A BONE

(76) Inventor: Gernot Teichmann, Duesseldorf (DE)

Correspondence Address: MATTHIAS SCHOLL, 14781 MEMORIAL DRIVE, SUITE 1319, HOUSTON, TX, 77079, US

(21) Appl. No.: 12/741,205

(22) PCT Filed: Sep. 19, 2008
(86) PCT No.: PCT/DE2008/075006

§ 371 Date: May 4, 2010

(30) Foreign Application Priority Data
Nov. 2, 2007 (DE) 20 2007 015 345.7

(51) Int. Cl. A61B 17/16

(52) U.S. Cl. 606/80

(57) ABSTRACT

Disclosed is a device (1) for creating a screw-shaped, especially a worm-shaped recess in a bone. In order to design a device (1) of said type which allows the recess to be quickly and safely introduced, a driving mechanism (3 to 9, 18 to 23) is proposed which applies torques (M1, M3) to the tool (13) via the shaft (9), said torques (M1, M3) extending in the direction of rotation of the tool (13). The first torque (M1) has an at least nearly constant quantity while the second torque (M3) is greater than the first torque (M1) and continuously changes the direction of rotation thereof when material is removed.

The invention relates to a device of the kind defined in detail in the preamble of claim 1.

German utility model DE 20 2006 012 830 U1 from the applicant proposed designing the screw-shaped anchoring part of a jaw implant with a pronounced worm shape and selecting the ratio of external diameter to internal diameter equal to or greater than 2. Moreover, it also proposed a device for creating a screw-shaped recess for receiving a screw-shaped and particularly a worm-shaped anchoring part of this kind. This tool can be provided with a material-removing device, for example in the form of teeth, whose helical direction of movement is briefly reversed. However, said publication does not explain how such a device is specifically constructed. This is the starting point for the invention.

Proceeding from the circumstances outlined above and from said document, the object of the invention is to make available a device of the type in question, by means of which the screw-shaped and preferably worm-shaped recess can be formed quickly and safely in a bone, particularly in a jaw bone.

To achieve this object, the invention provides said device with a special driving mechanism for the tool. According to the invention, the device is in the first instance specifically characterized by a driving mechanism with which two torques oriented in the direction of rotation of the tool are applied to the tool via the shaft, the first torque having an at least almost constant magnitude, and the second torque being greater than the first torque and continuously reversing its direction of rotation during the removal of material.

The device thus applies two torques to the tool. A first torque of the shaft serves to ensure the screw-shaped forward drive and guiding of the tool connected to the shaft. For this purpose, it suffices to apply to the shaft, and thus to the tool, a torque of a certain magnitude and constancy. Although this torque acts continuously on the tool with a practically constant magnitude via the shaft, it turns the shaft, and thus the tool, only when the second torque described below acts on the tool and the tool removes material. It is only the removal of material that creates room for the guiding and advance of the tool under the effect of the first torque, which now turns the shaft relatively slowly. The first torque can be controlled or regulated separately to its relatively low, constant value.

A second, greater torque of the shaft superimposes the first torque and serves to actually drive the tool which, by removing material, is intended to widen the cylindrical bore so as to form what is in principle a helical or at least partially helical recess. This purpose is served by a hammering movement, also here called an oscillating movement, of the tool, which movement can to a certain extent be modified, in terms of stroke and impact rate, by control or regulation of the second, relatively high torque.

On the basis of the drive concept according to the invention, the two torques required here can be controlled or regulated completely independently of each other. This means that the device is highly adaptable to the circumstances presented by each patient. In particular, the advance and drive of the tool can be adapted very precisely to the individual nature of the particular bone or the bone quality. This allows the recess to be produced quickly and safely and in a way that does not place any great strain on the patient.

The hammering, oscillating movement of the tool, which movement is generated by the reversal of the second torque, can extend over very short paths, calculated in fractions of a millimeter or tenth of a millimeter, and can be carried out at a high rate. Thus, depending on the external diameter of the recess for example, provision can be made for the reversal of the direction of rotation of the second torque to take place preferably at least 50 times or even 500 times per revolution of the shaft. These values also depend in particular on the type, shape and size of the tool and can, if appropriate, be adjusted within a certain range on the device, in order thereby to optimally adapt the particular tool to the individual and different circumstances of the particular case of use.

The first torque serves for the advance of the tool, during the preferably material-cutting work of the latter, and for the reverse stroke of the tool after the work. The first torque can have an adjustable and relatively small magnitude that remains constant within narrow limits, the second torque being preferably at least twice as great as the first torque. Here, within narrow limits means without great fluctuations, for example of not more than 20 to 50%, from the desired value. Here, relatively small generally means a fraction of the magnitude of the second torque, for example also a third or even less.

In one embodiment of the device, the tool is arranged on the shaft in a rotationally fixed but axially movable manner. A tool mounted in this way can be easily and quickly replaced by a new tool or by a tool of different dimensions, such that, with different tools being suitably kept in stock, the device can be used in a highly versatile or practically universal manner. The tool can be secured, for example, by virtue of the fact that the tool is connected to the shaft via what is in principle an axial groove-and-tongue guide. The groove-and-tongue guide can preferably be designed helically and with a large pitch. Here, large pitch means in particular a pitch of over 45°, preferably of over 60° to almost 90°. As the shaft turns, the helical shape generates an axially directed force component on the tool. This force component helps, on the one hand, in the formation of the screw-shaped recess and, on the other hand, in the removal of the tool after the recess has been completed.

In one embodiment of the device, the material-removing parts of the tool, or at least the bearing part thereof, are arranged helically or at least partially helically and with a small pitch. Material-removing parts can be teeth or the like. Here, small pitch means in particular a pitch of below 45°, but preferably of below 30° to just a few degrees.

If the two abovementioned helical arrangements are chosen, provision can then preferably be made that the helix of the groove-and-tongue guide is perpendicular to the helix of the tool, that is to say the axes of the two helixes are perpendicular to each other. For example, the pitches of the two helixes can be 80° and 10°.

In the design of the drive, provision can be made that the shaft is driven via a worm gear comprising a worm wheel, which is arranged on the shaft and is driven by a worm. In order to permit a small axial movement of the worm relative to the worm wheel, such a worm gear should preferably be designed as a cylindrical worm gear whose worm teeth have an optionally rounded trapezoidal profile.

To generate the two torques or movements of the tool, it is proposed that the worm is driven via a rotatable second shaft, which can move in an axially oscillating manner. The second shaft is driven by a first driving mechanism for applying the first torque, and by a second driving mechanism for applying an oscillating, axial movement of the second shaft. The axial, oscillating movement, i.e. reciprocating movement, of the second shaft, and thus of the worm, can take place over a very small distance depending on the step-down of the worm gear, which distance is, for example, only in the region of one millimeter or even less.

In order to remove the tool quickly and safely again from the screw-shaped recess after the latter has been completed, it is proposed that the direction of rotation of the torque of the first driving mechanism is reversible. Thus, by simply switching the direction of rotation of the first driving mechanism, the tool can be recovered from the finished recess, during which time the second driving mechanism is switched off. Since the tool, during this return stroke, simply has to travel along the already finished recess, the relatively small magnitude of the first torque is also sufficient for this purpose.

For the actual driving of the tool, the two driving mechanisms can comprise in particular an electric motor or a turbine. Such drives are also known from dentistry or surgery. In order to achieve a compact structure, the worm gear and the two driving mechanisms can be accommodated in a common housing. The device can be designed either as a very compact, manually operated tool or as a stationary device.

Further particular advantages, features and details of the invention will also be found in the following description of an illustrative embodiment.

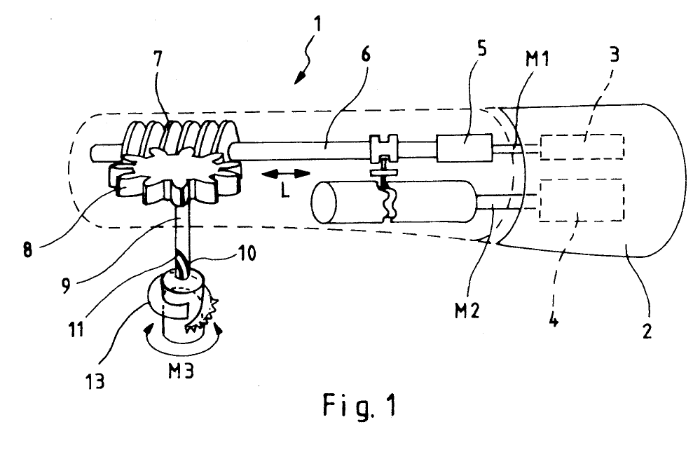

FIG. 1 shows an overall view of a device according to the invention, in a perspective and schematic depiction.

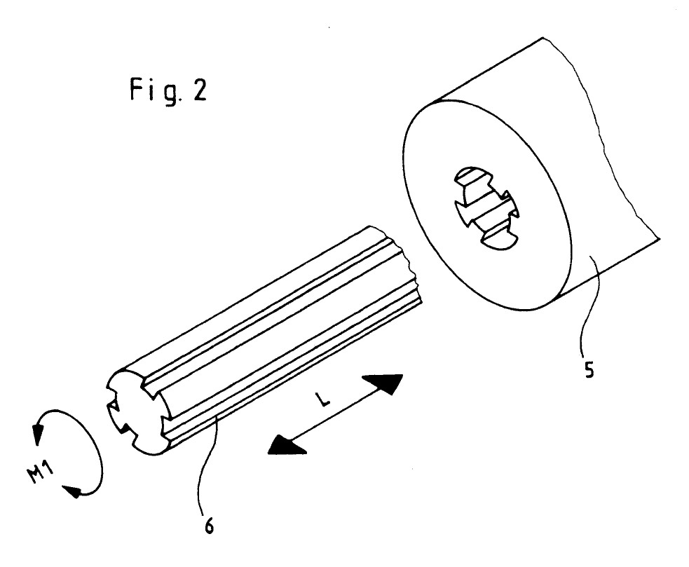

FIG. 2 shows a detail of a first drive.

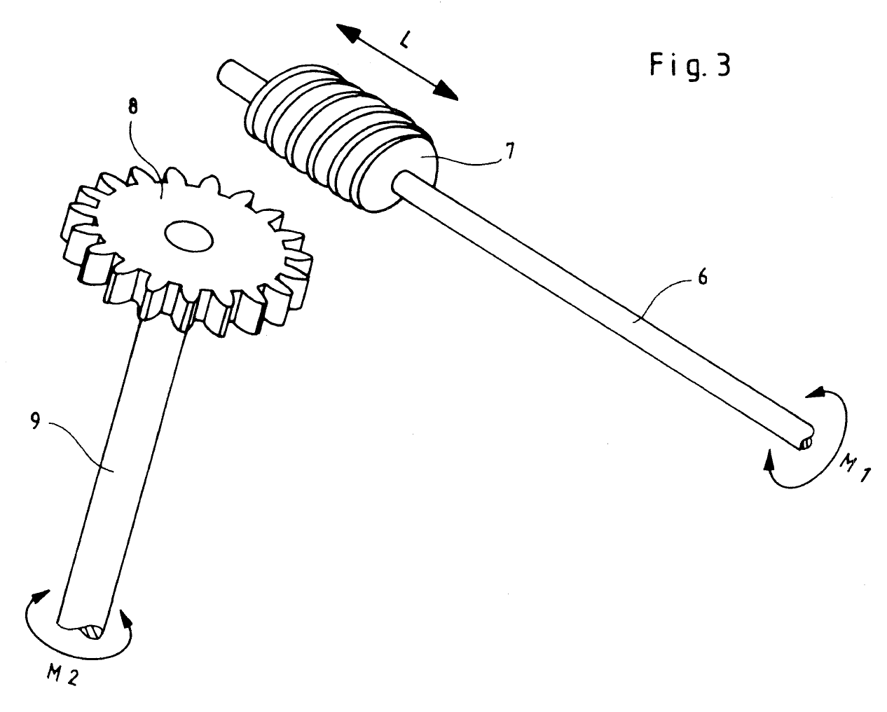

FIG. 3 shows a worm gear with a worm shaft and a tool shaft.

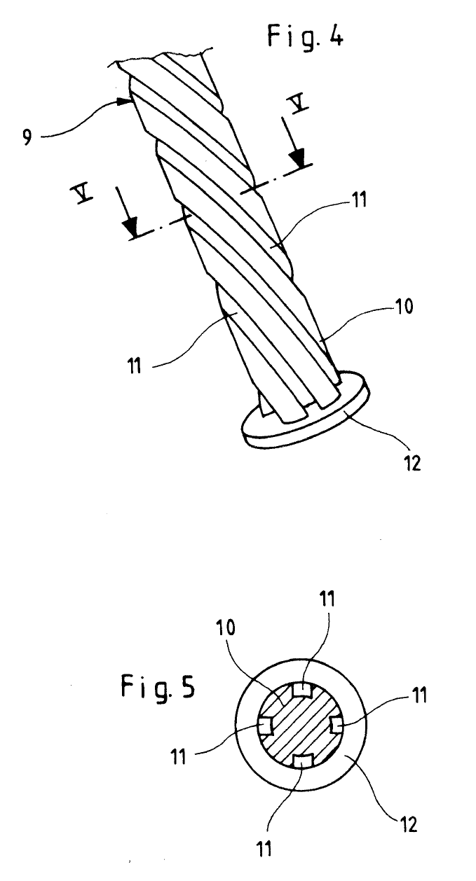

FIG. 4 shows the lower end of the tool shaft.

FIG. 5 shows the cross section V-V in FIG. 4.

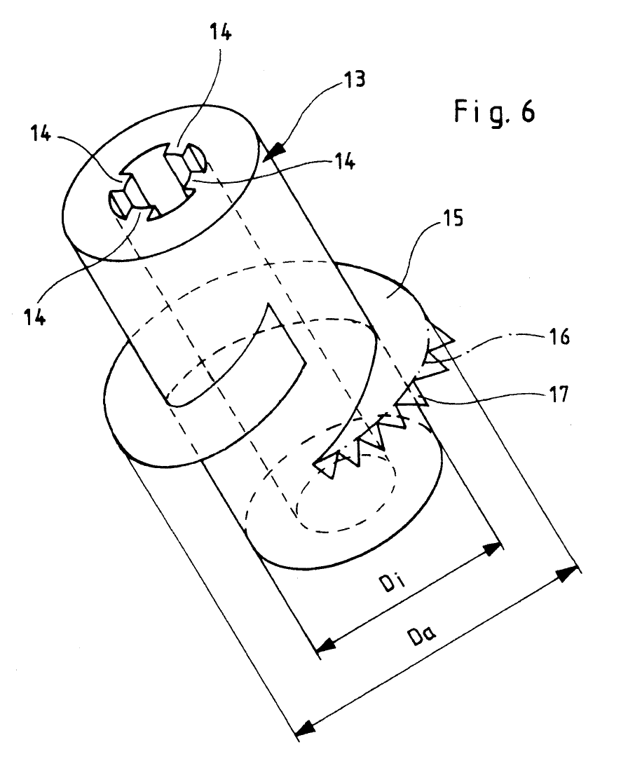

FIG. 6 shows a tool that is to be arranged on the lower end of the tool shaft.

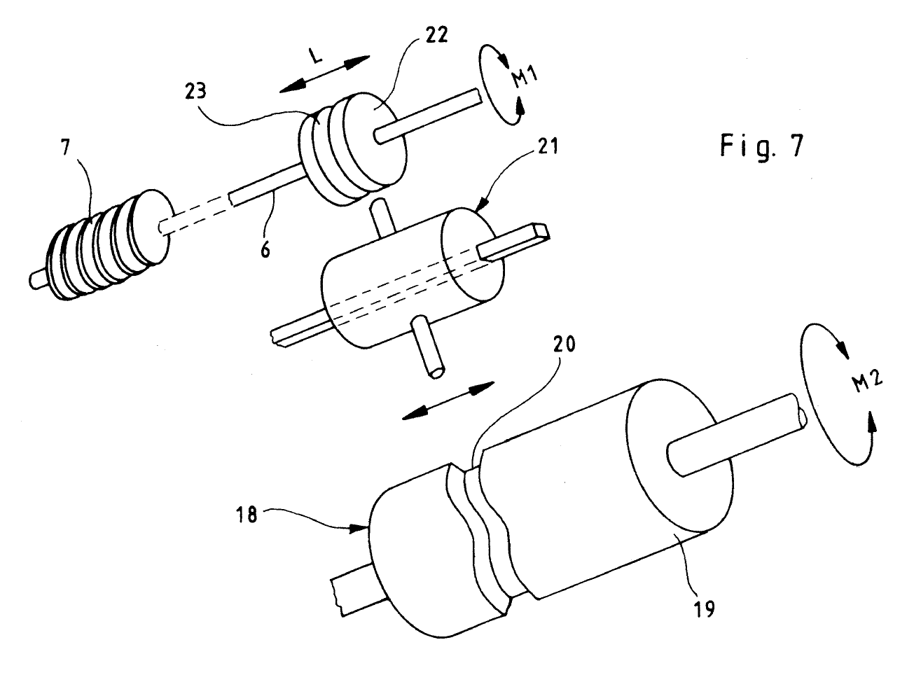

FIG. 7 shows the coordination of two drives on a common drive shaft.

The device 1 represents a hand-held surgical tool with the aid of which a blind bore in a jaw bone is to be widened to form a worm-shaped recess. A device of this kind, including the mode of action thereof, is described in principle in DE 20 2006 012 830 U1, and therefore only the novel construction and the novel mode of action of the device 1 are described here.

The device 1 comprises, according to FIG. 1, a maneuverable housing 2, in the rear part of which a first motor 3 for generating a first torque M1 and a second motor 4 for generating a second torque M2 are arranged. The front part of the housing 2 can be connected releasably to the rear part, and it is only symbolized here, so as to be able to better describe the components located therein.

The first motor 3 drives a hollow shaft 5 with the torque M1, in which hollow shaft 5, in the manner of a groove-and-tongue connection, a horizontal worm shaft 6 is mounted in such a way as to be movable to a limited extent, but rotationally fixed (compare FIG. 2). The worm shaft 6 is connected fixedly to a worm 7, which is designed as a cylindrical worm with a slightly rounded trapezoidal profile. The worm 7 meshes with a worm wheel 8, of which the shaft 9 is perpendicular and thus at right angles to the worm shaft 6 (compare FIG. 3).

At its lower section 10, the perpendicular shaft 9 is provided on the outside with a number of lengthwise grooves 11, which extend in a gentle helical formation in the jacket surface of the shaft 9. The lower section 10 ends with a flange 12 that protrudes radially out from the jacket surface (compare FIGS. 4 and 5).

A tool 13, which is pushed onto the lower section 10 of the shaft 9, is hollow (compare FIG. 6) and is able to engage, by means of inner helically protruding webs 14, in the grooves 11 of the shaft 9. The tool 13 is thus mounted on the shaft 9 in a rotationally fixed manner but can move along a gentle helical path in the longitudinal direction. Toward the bottom, the tool 13 bears on the flange 12.

The tool 13 comprises a tool carrier in the form of a radially protruding, helical rib 15, with an internal diameter Di and an external diameter Da. The rib 15 begins at the bottom with the internal diameter Di, which increases by way of an arc 16 to the external diameter Da, in order then to continue helically upward with the same diameter Da about approximately one revolution. In the area of the arc 16 from the internal diameter Di to the external diameter Da, several radially and axially protruding teeth 17 are arranged on the rib 15.

While the first motor 3 drives the worm shaft 6 with the first torque M1, the worm shaft 6 is also driven by the second motor 4 in the following way. The motor 4 drives a horizontal, stationary roller 18, which extends parallel to the worm shaft 6 and on the jacket surface 19 of which a sinusoidal circumferential groove 20 is formed. A double cam 21 guided lengthways to a limited extent is arranged parallel to the roller 18. Parallel to the double cam 21, an annular slide piece 22 is connected fixedly to the worm shaft 6 and has, on the outside, a straight annular groove 23. The double cam 21 engages with play in the groove 20, on the one hand, and in the groove 23, on the other hand.

When the tool 13 is introduced into the bore (not shown) in the jaw bone, the two motors 3 and 4 are started up, which has the following effect. The motor 3 applies a torque M1 to the worm shaft 6, then to the worm 7, then to the worm wheel 8, then to the shaft 9, then to the tool 13, as a result of which, finally, the teeth 17 are as it were fed forward by the torque M1.

To ensure that the teeth 17, which tend more or less radially forward under the torque M1, are brought into a hammer-like movement, a torque M2 is at the same time applied to the roller 18 by the motor 4. The rotating roller 18 converts the torque M2 via the double cam 21 and the slide piece 22 into a lengthwise movement L of the worm shaft 6, which in turn slightly moves the worm 7 axially. By means of the axial movement of the worm 7, a torque M3 is generated on the worm wheel 8 meshing with the worm 7, which torque M3 is transferred to the shaft 9 and from there to the tool 13 and then acts in addition to the torque M1 on the tool 13.

Since the lengthwise movement L oscillates, worm wheel 8, shaft 9 and tool 13 accordingly turn to and fro under the torque M3, in other words continuously change their direction of rotation at a defined rate. As a result, an approximately tangential, hammering movement occurs on the teeth 17, which movement superimposes the torque M1. Consequently, under the effect of the two torques M1 and M3, the teeth 17 of the tool 13 can work their way very quickly around the bore into the jaw bone and complete the helical recess.

### LIST OF REFERENCE SIGNS

1 device
2 housing
3 motor
4 motor
5 hollow shaft
6 worm shaft
7 worm
8 worm wheel
9 shaft
10 section
11 groove
12 flange
13 tool
14 web
15 rib
16 arc
17 tooth
18 roller
19 jacket surface
20 groove
21 double cam
22 slide piece
23 groove
Di internal diameter
Da external diameter
L lengthwise movement
M1 torque
M2 torque
M3 torque

1. A device (1) for creating a screw-shaped, particularly worm-shaped recess in a bone in order to receive a screw-shaped, particularly worm-shaped anchoring part of a bone implant, with a tool (13) which is driven by a shaft (9) and removes material and with which a cylindrical bore can be widened to form an at least partially helical recess, characterized by a driving mechanism (3 to 9, 18 to 23) with which two torques (M1, M3) oriented in the direction of rotation of the tool (13) are applied to the tool (13) via the shaft (9), the first torque (M1) having an at least almost constant magnitude, and the second torque (M3) being greater than the first torque (M1) and continuously reversing its direction of rotation during the removal of material.

2. The device (1) as claimed in claim 1, characterized in that the reversal of the direction of rotation of the second torque (M3) occurs at least 50 times per revolution of the shaft (9).

3. The device (1) as claimed in claim 2, characterized in that the reversal of the direction of rotation of the second torque (M3) occurs at least 500 times per revolution of the shaft (9).

4. The device (1) as claimed in one of claims 1 through 3, characterized in that the second torque (M3) is at least twice as great as the first torque (M1).

5. The device (1) as claimed in one of claims 1 through 4, characterized in that the tool (13) is arranged on the shaft (9) in a rotationally fixed but axially movable manner.

6. The device (1) as claimed in claim 5, characterized in that the tool (13) is connected to the shaft (9) via an axial groove-and-tongue guide (11, 14).

7. The device (1) as claimed in claim 6, characterized in that the groove-and-tongue guide (11, 14) is designed helically and with a large pitch.

8. The device (1) as claimed in one of claims 1 through 7, characterized in that the material-removing parts (17) of the tool (13), or at least the bearing part (15) thereof, are arranged helically or at least partially helically and with a small pitch.

9. The device (1) as claimed in claims 7 and 8, characterized in that the helix of the groove-and-tongue guide (11, 14) is perpendicular to the helix of the tool (13).

10. The device (1) as claimed in one of claims 1 through 9, characterized in that the shaft (9) is driven via a worm gear (7, 8).

11. The device (1) as claimed in claim 10, characterized in that the worm gear (7, 8) comprises a worm wheel (8), which is arranged on the shaft (9) and is driven by a worm (7).

12. The device (1) as claimed in claim 11, characterized in that the worm (7) is driven via a second shaft (6), which is rotatable and can be moved in an axially oscillating manner.

13. The device (1) as claimed in claim 12, characterized in that the second shaft (6) is driven by a first driving mechanism (3, 5) for applying the first torque (M1), and by a second driving mechanism (4, 18 to 23) for applying an oscillating, axial movement (L) of the second shaft (6).

14. The device (1) as claimed in one of claims 1 through 13, characterized in that the direction of rotation of the torque (M1) of the first driving mechanism (3, 5) is reversible.

15. The device (1) as claimed in claim 13 or 14, characterized in that the two driving mechanisms (3, 4) comprise an electric motor or a turbine.

16. The device (1) as claimed in one of claims 13 through 15, characterized in that the worm gear (6 to 9) and the two driving mechanisms (3, 4, 5, 18 to 23) are accommodated in a common housing (2).

17. The device (1) as claimed in one of claims 1 through 16, characterized in that the device (1) is designed as a manually operated tool.

18. The device as claimed in one of claims 1 through 16, characterized in that the device is designed as a stationary device.
